(19) United States
(12) Patent Application Publication
(10) Pub. No.: US 2015/0196685 A1
(43) Pub. Date: Jul. 16, 2015

(54) FUNCTIONALIZED SUBSTRATES AND METHODS OF MAKING SAME

(71) Applicant: PRINCETON UNIVERSITY, Princeton, NJ (US)

(72) Inventors: Jeffrey Schwartz, Princeton, NJ (US); Thomas J. Dennes, Parkesburg, PA (US)

(21) Appl. No.: 14/662,925

(22) Filed: Mar. 19, 2015

Related U.S. Application Data
(63) Continuation of application No. 12/253,462, filed on Oct. 17, 2008.
(60) Provisional application No. 60/960,859, filed on Oct. 17, 2007.

(51) Int. Cl. A61L 27/30; A61L 27/18; C23C 18/12

(52) U.S. Cl. CPC A61L 27/306; C23C 18/1216; C23C 18/1233; A61L 27/18

(57) ABSTRACT

Polymer substrates including adhesion layers for activating the surface of the substrate are provided, thereby allowing the substrate to react with organic, inorganic, metallic and/or organometallic materials. The surface of the polymer substrate is coated with a metal oxide layer that is subjected to conditions adequate to form an oxide adhesion layer. Combining deposition techniques for formation of functionalized polymer surfaces with photolithographic techniques enables spatial control of RGD presentation at the polymer surfaces are achieved with sub-cellular resolution. Surface patterning enables control of cell adhesion location at the surface of the polymer and influences cell shape. Metallization of polymers as described herein provides a means to prepare metal-based electrical circuitry on a variety of flexible substrates.

### CROSS-REFERENCE TO RELATED APPLICATIONS

This application is a continuation of U.S. patent application Ser. No. 12/253,462 filed Oct. 17, 2008 and claims the benefit of U.S. Provisional Patent Application Ser. No. 60/960,859, filed Oct. 17, 2007, the entireties of which are incorporated herein by reference.

### STATEMENT REGARDING FEDERALLY SPONSORED RESEARCH

This invention was made with government support under Grant No. CHE-0612572 awarded by the National Science Foundation. The Government has certain rights in this invention.

### FIELD OF THE INVENTION

The present invention relates to substrates with activated surfaces. In particular, a thin layer of metal oxide on the surface of a polymer substrate forms an adhesion layer for activating the surface of the substrate.

### BACKGROUND OF THE INVENTION

An activated layer which is bonded to the surface of a substrate is useful in making devices for use as an interface between the substrate and other materials such as organic or metallic materials. This activated layer allows the substrate to react with and to bind to the organic or metallic material.

It is well known to develop an organic layer covering a substrate surface using polymerization methods to deposit a polymer layer on the surface. In general these layers do not display good surface conformation. That is, the growth of polymer overlayers tends to occur by surface attachment of monomer moieties at isolated locations across the surface (“islands”) which are incorporated into a polymer as polymerization proceeds from these islands outward until the polymeric mass eventually bridges over the surface between “islands”, but without forming additional chemical bonds to the surface between the “islands”. This growth and bonding pattern tends to form layers which have a thickness equal to many layers of the species comprising the layer (often hundreds of nanometers to microns thick) but which have relatively few bonds between the species comprising the coating layer and the substrate surface.

Organic layers comprising bulk polymers, applied for example, by “spin-on” techniques are also well known. These types of coatings also display the same sparse bonding pattern between the substrate surface and the coating. Coated substrates having a low number of bonds per unit area of surface between the coating and substrate surface exhibit poor mechanical attachment between the substrate and the coating and poor electronic communication between the substrate surface and the coating. As a consequence they are not mechanically robust and do not in general display long term stability. Such coatings also may not display efficient charge carrying properties when used in electronic devices. An example of organic monolayers on inorganic substrates is found is U.S. Pat. No. 6,146,767, the entirety of which is incorporated herein by reference.

In some applications, it may be necessary to activate the substrate surface before depositing subsequent layers on the surface. The present inventors have previously shown that it is possible to functionalize substrates that contain acidic protons, such as —OH or —NH groups, by their reaction with Group IV alkoxides. This procedure yields a molecular adhesion species that is bound to the surface of the bulk polymer, but is limited to materials that have acidic groups on their surface. See, Dennes, T. J.; Hunt, G. C.; Schwarzbauer, J. E.; Schwartz, J. High-Yield Activation of Scaffold Polymer Surfaces to Attach Cell Adhesion Molecules. J. Am. Chem. Soc. 2007, 129, 93-97 (p. 95, below Scheme 3, col. 2, lines 20-38; p. 96, below FIG. 1, col. 1 lines 1-24 and col. 2 lines 1-6); and Dennes, T. J.; Schwartz, J. Controlling cell adhesion on polyurethanes. Soft Matter 2008, 4, 86-89 (pg.87, below Scheme 1, col. 1 lines 22-24 and col. 2 lines 1-19), incorporated herein by reference. Thus biomedically important polyesters and polyketones, which do not have readily acidifiable groups, cannot be employed. Additionally, the adhesion species are individual molecules attached to a polymer surface and do not form a continuous matrix that coats the surface. To that end, it is desirable to functionalize the surface of a polymer substrate with a continuous, thin alkoxide layer that does not require a proton transfer step.

### SUMMARY OF THE INVENTION

The present invention provides a broadly applicable chemical process for activation not only of polyamides and polyurethanes, but also polyesters, polyketones, polyethers, polyimides, aramides, polyfluoroolefins, epoxies, or composites containing these polymers.

In one embodiment, the present invention provides activated, or functionalized, polymer surfaces that can be used to covalently bond subsequent material or layers thereof on the surface. The polymer is coated with a thin layer of metal oxide (oxide adhesion layer) in what may be termed a continuous layer. “Continuous layer” as used herein refers to a layer that is formed by a matrix of individual molecules that are chemically bonded and linked to each other, as opposed to individual molecules covering the surface. In the present case, metal alkoxide molecules are bonded together on at least a portion of a polymer surface to form a continuous layer. One major advantage of a continuous layer is that the entirety of the surface that is covered by the continuous metal oxide adhesion layer is activated. In the prior art, where individual molecules are laid on the surface, only the area of the surface where an acidic proton is available, i.e., the area with acidic functionality, can be activated.

In accordance with one aspect of the present invention, a polymer surface may include acidic functionality regions as well as regions coated with a metal alkoxide functionalized layer. In such embodiments the metal alkoxide functionalized layer may be viewed as filling in the spaces between the regions of acidic functionality.

In accordance with another embodiment metal alkoxide functionalized layers may be applied to regions of polymer having acidic functionality.

The metal oxide adhesion layer is thin, about 1 nm-1 μm, preferably about 2 nm, such that it is flexible. The thin layer allows the oxide adhesion layer to bend with the substrate material without cracking, peeling, or breaking.

The coating process involves depositing a metal alkoxide on the polymer, and heating the substrate, with or without partial hydrolysis, so that the metal alkoxide molecules form a continuous metal oxide adhesion layer covalently attached to the polymer surface.

Various polymer surfaces, including surfaces of polyethylene terephthalate (PET) and polyetheretherketone (PEEK), can be functionalized via an alkoxide adhesion layer. By reaction with the adhesion layer RGD-terminated polymer surfaces were prepared and achieved the highest loadings yet reported on polymers (40-180 pmol/cm² or 10-40% spatial coverage) and were successful for enabling attachment and spreading of fibroblasts or osteoblasts in vitro. When vapor-deposition techniques for formation of functionalized polymer surfaces are combined with known photolithographic techniques, spatial control of RGD presentation at the polymer surfaces are achieved with sub-cellular resolution. This surface patterning enables control of cell adhesion location at the surface of the polymer and influences cell shape. Metallization of polymers in accordance with the present invention provides a means to prepare metal-based electrical circuitry on a variety of flexible substrates.

### BRIEF DESCRIPTION OF THE DRAWINGS

A more complete understanding of the invention may be obtained by reading the following description of specific illustrative embodiments of the invention in conjunction with the appended drawings in which:

FIG. 1 depicts a schematic exemplary method for activating the surface of a polymer to achieve a composition in accordance with at least one aspect of the present invention;

FIG. 2 depicts a schematic exemplary method for making a composition in accordance with at least one aspect of the present invention;

FIG. 3 depicts a schematic exemplary method for making a composition in accordance with at least one aspect of the present invention;

FIG. 4 depicts a schematic exemplary method in accordance with at least one aspect of the present invention;

FIG. 5 depicts a schematic exemplary method in accordance with at least one aspect of the present invention;

FIG. 6 depicts a schematic exemplary method of formation of a metal oxide/alkoxide layer on a polymer surface in accordance with at least one aspect of the present invention;

FIG. 7 depicts a schematic exemplary method of phosphonic acid deposition on an adhesion layer in accordance with at least one aspect of the present invention;

FIGS. 8A and 8B are graphical depictions of X-ray photoelectron spectra (XPS) of phosphorous (P) regions (FIG. 8A) and zirconium (Zr) regions (FIG. 8B) of an organophosphonate bound to the adhesion layer on PET in accordance with at least one aspect of the present invention;

FIGS. 9A and 9B depict atomic force micrograph (AFM) images of PET (FIG. 9A) and phosphonic acid on PET (FIG. 9B) in accordance with at least one aspect of the present invention;

FIG. 10 depicts an exemplary schematic method of binding carboxylic acids and silanes to PET and PEEK via an adhesion layer in accordance with at least one aspect of the present invention;

FIGS. 11A-11D depict images of osteoblast cell attachment on derivatized PEEK in accordance with at least one aspect of the present invention. FIG. 11A depicts cells on RGD-modified PEEK (30a), FIG. 11B depicts C₁₂bisphosphonate (C12BP)-modified PEEK, and FIG. 11C PEEK control surfaces after 3 h, fixed and stained with anti-vinculin antibodies and fluorescein-conjugated secondary antibodies. Scale bars are 50 μm. FIG. 11D indicates then number of cells per 10× microscope field counted for untreated PEEK, RGD-derivatized, and C12BP-derivatized PEEK;

FIG. 12 is a schematic depiction of a method of patterning fluorescein or RGD onto PET and PEEK in accordance with at least one aspect of the present invention;

FIGS. 12A and 12B are images of patterned fluorescein according to the method depicted in FIG. 12 on PEEK (FIG. 12A) and PET (FIG. 12B) in accordance with at least one aspect of the present invention (scale bars are 50 μm);

FIG. 13 is an image of patterned rhodamine (red-background) and fluorescein (green-circles) on PET in accordance with at least one aspect of the present invention (scale bar is 50 μm);

FIGS. 14A-14C are images of cells seeded on RGD islands on Nylon 6/6 (FIGS. 14A and 14B) and PET (FIG. 14C) in accordance with at least one aspect of the present invention. Cells were stained for vinculin-containing focal adhesions in FIGS. 14A and 14B) and labeled with Cell Tracker Green® in FIGURE C. Red circles indicate pattern boundaries in FIG. 14C (scale bars are 50 μm);

FIGS. 15A and 15B are graphical depictions of electron dispersive X-ray (EDX) analysis before (FIG. 15A) and after (FIG. 15B) reduction of a copper salt bound to the adhesion layer with dimethylamineborane (DMAB) in accordance with at least one aspect of the present invention;

FIGS. 16A and 16B are images of EDX maps of Zr (FIG. 16A) and Cu (FIG. 16B) features patterned on Kapton® polyimide film in accordance with at least one aspect of the present invention; and

FIGS. 17A and 17B are graphical representations of AFM of Cu “seed” patterned on Kapton® (a registered trademark of DuPont) polyimide film in 10 μm features by DMAB reduction (FIG. 17A) and AFM of copper-filled “pits” formed by NaBH₄ reduction (FIG. 17B) in accordance with at least one aspect of the present invention.

It should be noted that the appended drawings illustrate only typical embodiments of this invention and are therefore not to be construed as limiting of its scope, for the invention may admit to other equally effective embodiments. Where possible, identical reference numerals have been inserted in the figures to denote identical elements.

### DETAILED DESCRIPTION OF THE PREFERRED EMBODIMENTS

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

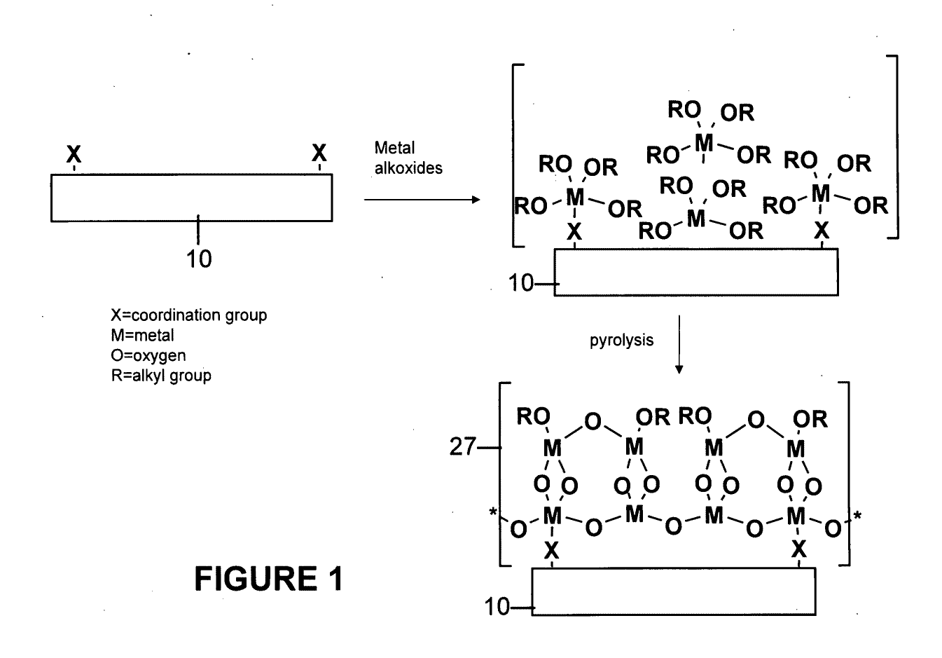

Now referring to FIG. 1 devices or compositions in accordance with the present invention include a surface activated polymer substrate 10 having coordination groups X, and an oxide adhesion layer 27 bonded to a surface thereof via coordination groups X, wherein the oxide adhesion layer 27 is a metal alkoxide generally depicted as M-O-R. The oxide adhesion layer 27 is one that has been subjected to a process such as but not limited to pyrolysis, microwaving, complete hydrolysis and/or partial hydrolysis.

Polymeric substrate 10 is any polymer that can be functionalized, and may include any of various substances comprising synthetic and/or natural polymer molecules having a surface coordinating group X capable of coordinating with the metal atom M of the metal alkoxide. Examples of suitable polymer substrates include, but are not limited to, polyamides (e.g., proteins), polyurethanes, polyureas, polyesters, polyketones, polyimides, polysulfides, polysulfoxides, polysulfones, polythiophenes, polypyridines, polypyrroles, polyethers, silicones (polysiloxanes), polysaccharides, fluoropolymers, epoxies, aramides, amides, imides, polypeptides, polyethylene, polystyrene, polypropylene, glass reinforced epoxies, liquid crystal polymers, thermoplastics, bismaleimide-triazine (BT) resins, benzocyclobuteneABFG×13, low coefficient of thermal expansion (CTE) films of glass and epoxies, and composites including these polymers. Essentially, any donor-electron pair on the surface of the polymer capable of coordinating with the metal alkoxide is suitable for use with the present invention. In preferred embodiments the polymer substrates are polyethylene terephthalate (PET), polyetheretherketone (PEEK), polyimides, aramides, epoxies, and nylon. The oxide adhesion layer 27 adheres to the surface of the polymer by the covalent bonding between the coordinating group on the surface of the polymer and the metal of the metal alkoxide.

Alkoxides of transitional metals are particularly useful for the present invention. Periodic Table Group 3-6 and 13-14 metals are desirable metals for compositions of the present invention. The preferred metals are Zr, Al, Ti, Hf, Ta, Nb, V and Sn, with the most preferred metals being Zr and Ta. Depending upon the position of the transition metal on the Periodic Table, the transition metal alkoxide will have from three to six alkoxide groups or a mixture of oxo and alkoxide groups. Preferred alkoxide groups have from 2 to 4 carbon atoms, such as ethoxide, propoxide, iso-propoxide, butoxide, iso-butoxide, tert-butoxide and fluoronated alkoxide. The most preferred metal alkoxides are zirconium tetra(tert-butoxide) and tantalum pentaethoxide.

Compositions in accordance with the present invention may include additional material bound via the oxide adhesion layer 27 to the polymer substrate 10. Such additional material includes but is not limited to organic, metallic, organometallic or inorganic compounds. The usefulness of the additional material will be apparent to those skilled in the art. For example, further organic material can be used in making biosensors, gene chips and the like, while metals can be laid to make semiconductor chips, flexible electronic devices and circuits or the like. Further organometallic material can be used in making supported catalysts, synthetic reagents and the like, while inorganic materials can be laid down to make seed beds for electroless metal deposition, and antibacterial coatings or the like.

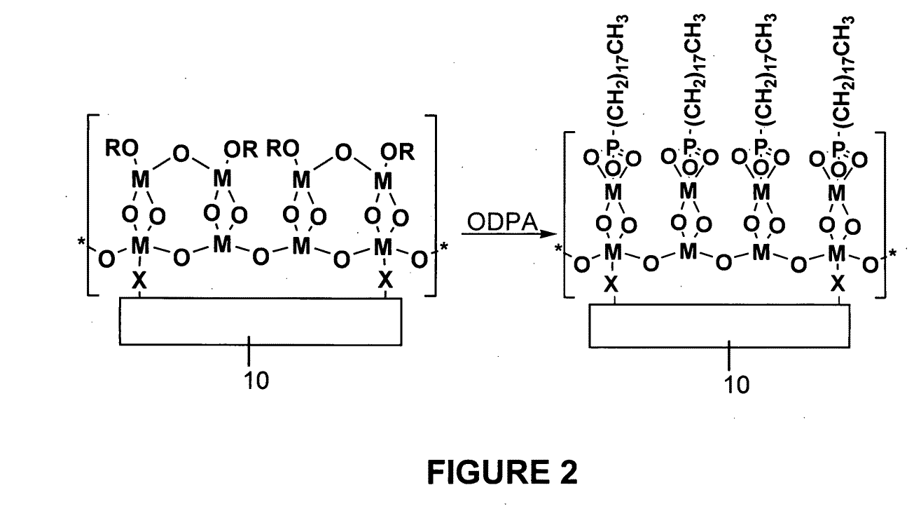

Suitable further organic materials, compounds or complexes include but are not limited to organic compound sufficiently acidic to react with a metal oxide or alkoxide; carboxylic, phosphonic, phosphoric, phosphinic, sulfinic, sulfonic and hydroxamic compounds, nucleic acids, polymers, proteins, organic acids, and the like. Now referring to FIG. 2, for example, the additional material is an organic compound octadecylphosphonic acid (ODPA), which forms a bond as octadecylphosphonate with oxide adhesion layer 27.

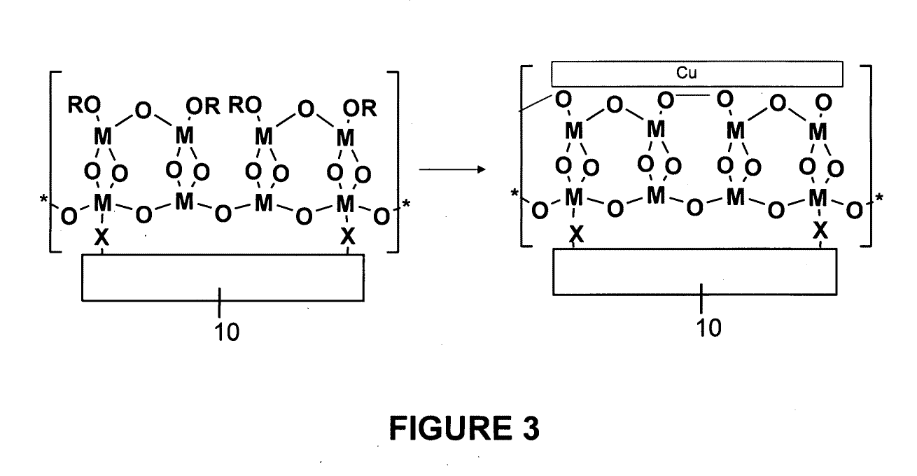

Suitable further metallic materials, compounds or complexes include but are not limited to copper, silver, gold, aluminum, nickel, palladium, rhodium and platinum and salts thereof. Now referring to FIG. 3, copper is exemplified as an additional material.

Suitable further organometallic materials, compounds or complexes include but are not limited to organometallic compounds that can react with an oxide, alkoxide, hydroxide or hydroxyl. Examples include but are not limited to alkyls, alkoxides, amides, substituted amides, complexes containing ligands comprising acidic functional groups including phosphonic, carboxylic, phosphinic, hydroxamic, and sulfonic acids.

Suitable further inorganic materials, compounds or complexes include but are not limited to inorganic materials with a high dielectric constant, silanes, siloxanes, carboxylates, phosphonates, alkenes, alkynes, alkyl halides, epoxides, carboxylic esters, amides, phosphonate ester and imides.

The additional material may be introduced to the oxide adhesion layer by techniques know to those of skill in the art, including but not limited to evaporative, sputter, immersion or extractive deposition. In some embodiments it may be desirable to subject the oxide adhesion layer to complete or partial hydrolysis prior to deposition of the additional material. In some embodiments it may be desirable to subject the deposited additional material to heat or microwave treatment.

The adhesion layer may be functionalized to elicit a biological response, such as but not limited to cell attraction, cell non-adhesion, and cell death by selecting a material for use with a substrate in a biological application. Suitable materials include saccharides, oligosaccharides, polysaccharides, organic acids, nucleic acids, proteins, and peptides.

Compositions and devices in accordance with the present invention may form or be included in various devices, including but not limited to cardiovascular implant devices, such as but not limited to stents, replacement heart valves (leaflets, sewing cuffs, and orifice), annuloplasty rings, pacemakers, pacemaker polymer mesh bags, pacemaker leads, pacing wires, intracardiac patches/pledgets, vascular patches, vascular grafts, defribillators, and intravascular catheters; tissue scaffold devices including but not limited to non-woven meshes, woven meshes, and foams; stents; and bone, joint and spinal implants; bone fixation cerclage; dental and maxillofacial implants; and other devices that would benefit from increased osteoconductivity; neurosurgical devices and implants such as but not limited to shunts and coils; and general surgical devices and implants such as but not limited drainage catheters, shunts, and vascular patches. Specifically, such devices may includes an embodiment of the present invention whereby the adhesion layer is functionalized to increase osteoconductivity. Examples of suitable materials/functionalized regions include for example polyetheretherketone (“PEEK”), nylon, polyethylenes, PET, polyurethanes, and silk.

Other devices of the present invention include compositions and materials described herein and further including at least some regions of in the oxide adhesion layer that are functionalized for bioresistance. In one embodiment the oxide adhesion layer is functionalized to include at least one polyethylene glycol bound (PEGylated) region, as is described in further detail hereinbelow in the Experiments.

Compositions and devices in accordance with this embodiment include but are not limited to all devices specific to an application of use by an orthopedic, cardiovascular, plastic, dermatologic, general, maxillofacial or neuro surgeon or physician including, but not limited to, diagnostic implant devices, biosensors, stimulators, diabetic implants such as glucose monitoring devices, external fixation devices, external fixation implants, orthopedic trauma implants, implants for use in joint and spinal disorders/reconstruction such as plates, screws, rods, plugs, anchors, bone anchors, cages, scaffolds, artificial joints (e.g., hand, wrist, elbow, shoulder, spine, hip, knee, ankle), wires and the like, oncology related bone and soft tissue replacement devices, dental and oral/maxillofacial devices, cardiovascular implants such as stents, catheters, valves, rings, implantable defibrillators, and the like, contact lenses, ocular implants, keratoprostheses, dermatologic implants, cosmetic implants, implantable medication delivery pumps; general surgery devices and implants such as but not limited to drainage catheters, shunts, tapes, meshes, ropes, cables, wires, sutures, skin staples, staples, soft tissue anchors, burn sheets, and vascular patches; and temporary/non-permanent implants.

In accordance with another embodiment the adhesion layer may be disposed on the substrate in a pattern or micropattern as described in further detail hereinbelow.

In accordance with another embodiment the additional material may be disposed on the adhesion layer in a pattern or micropattern as described in further detail hereinbelow.

The adhesion layer and/or the additional material contain at least two different regions of functionalization.

Methods of making compositions and devices in accordance with the present invention include activating a polymer surface comprising the steps of a) contacting a metal alkoxide with the surface; and b) subjecting the metal alkoxide to conditions adequate to form an oxide adhesion layer on the surface. The contacting step may be achieved by any suitable technique known to those skilled in the art such as but not limited to vapor or immersion deposition. The step of forming an oxide adhesion layer may be achieved by subjecting the metal alkoxide to conditions of pyrolysis, microwaving, complete hydrolysis or partial hydrolysis. When heating conditions are employed, it is preferred that the metal alkoxide is heated to between about 50° C. and the melting point of the polymer.

Now referring to FIG. 1, a schematic of one embodiment of the present invention for activating the surface of a polymer is depicted. A polymeric substrate 10 is functionalized according to the method of the present invention by coating at least a surface of the substrate 10 with a thin, continuous layer of metal alkoxide. The molecules of metal alkoxide are first brought into reactive proximity to the polymer molecules such as by, but not limited to, vapor deposition or immersion deposition methods known in the art. If an ultrathin layer is desired, vapor deposition is the preferred process. The deposited metal alkoxide molecules are then heated to between about 50° C. and the melting point of the polymer (the heating should not be at or above the melting point of the polymer) to pyrolyze the metal alkoxides. During pyrolysis, the individual metal alkoxide molecules are covalently bonded together forming a continuous metal oxide adhesion layer. It should be appreciated that, although FIG. 1 shows tetraalkoxides, other metals form different alkoxides. For example, transition metals of Groups 3 and 13 form trialkoxides; transition metals of Group 5 form pentaalkoxides or mixed oxoalkoxides; and transition metals of Group 6 form hexaalkoxides or mixed oxoalkoxides.

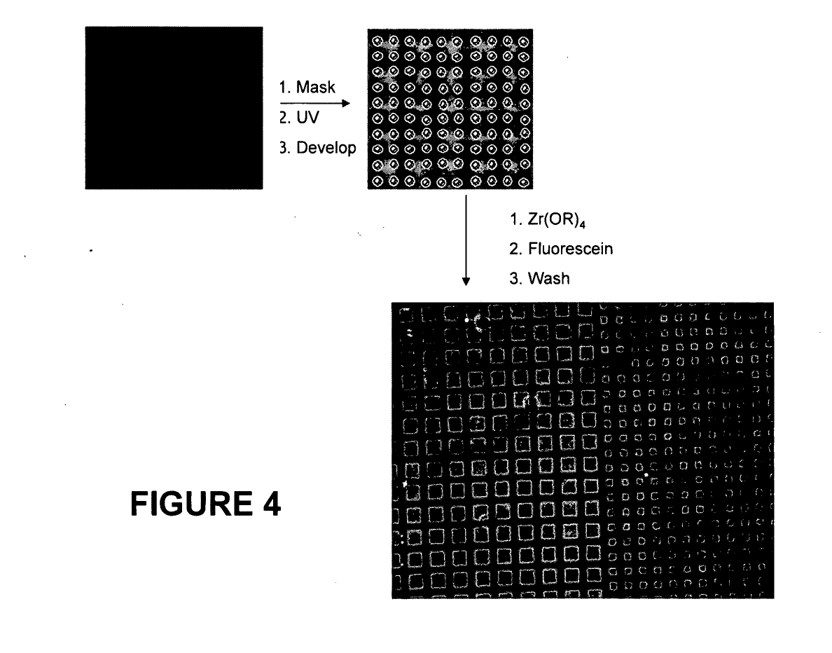

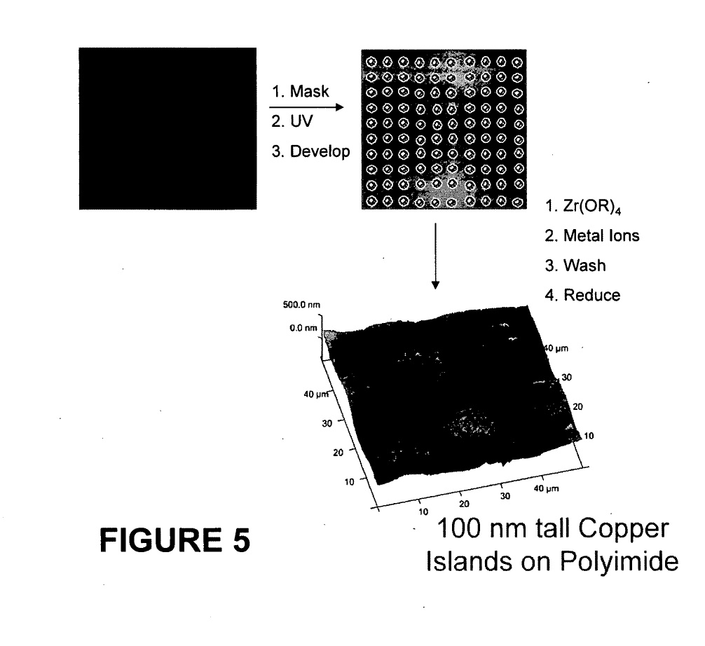

In accordance with another embodiment of the present invention, a further step may include reacting the oxide adhesion layer with an additional material selected from an organic, metallic, organometallic or inorganic compound to bind the additional material to the polymer surface via the oxide adhesion layer. The additional material may be added by reaction with the oxide adhesion layer by various methods available in the art, such as but not limited to evaporative, sputter, immersion or extractive deposition. In one embodiment of the present invention, the material may be added using lithography to lay a pattern of material on to the oxide adhesion layer. Now referring to FIGS. 4 and 5, a lithographic process is depicted for use with the present invention. The polymer surface is completely coated with a photoresist, and is then exposed to UV light through a mask. The areas that were exposed to the UV light can be developed and removed away, leaving openings in the photoresist and access to the polymer surface in small areas. These areas are functionalized with the metal oxide adhesion layer. The photoresist is then dissolved away in acetone leaving small patterned areas in the polymer surface that include the adhesion layer. The patterned areas are preferentially reactive toward organic compounds (FIG. 4) and metallic species (FIG. 5).

In accordance with one embodiment, the oxide adhesion layer may be subjected to complete or partial hydrolysis prior to deposition of the additional material to give the oxide adhesion layer with one or more alkoxide groups remaining on the metal atoms. In other embodiments, the deposited additional material is subjected to heat or microwave treatment to give the oxide adhesion layer with one or more alkoxide groups remaining on the metal atoms.

### EXAMPLES

Without further description, it is believed that one of ordinary skill in the art can, using the preceding description and the following illustrative examples, make and utilize the compounds and articles of the present invention and practice the claimed methods. The following examples are given to illustrate the present invention. It should be understood that the invention is not to be limited to the specific conditions or details described in these examples.

### Example 1

### Formation of a Zirconia Thin Film on Polymer Substrate

All reagents were obtained from Aldrich and were used as received unless otherwise noted. PET, PEEK, and nylon 6/6 were obtained from Goodfellow, Inc. Acetonitrile was dried over CaH₂; and tetrahydrofuran (THF) was dried over KOH overnight. Both were distilled prior to use. Surface modified samples were analyzed using a Midac M25 10C interferometer equipped with a surface optics SOC4000 SH specular reflectance head attachment. Fluorimetry experiments utilized a Photon Technology International Fluorescence Spectrometer.

Polymer substrates (nylon 6/6, PET or PEEK) were placed in a deposition chamber equipped with two stopcocks for exposure either to vacuum or to the vapor of zirconium tetra(tert-butoxide). The chamber was evacuated at 10⁻³ torr for 1 hour and polymer slides were exposed to vapor of zirconium tetra(tert-butoxide) (with external evacuation) for 1 minute followed by 5 minutes exposure without external evacuation. This cycle was repeated twice, after which a heating tape was applied to the chamber, and the internal temperature of the chamber was raised to 60° C. and kept at that temperature for 5 minutes (without external evacuation). The chamber was then allowed to cool and was then evacuated at 10⁻³ torr for 1 hour to ensure removal of excess zirconium tetra(tert-butoxide) and to give surface activated polymers (with reference to FIG. 1, where M=Zr, and R=tert-butyl). AFM section analysis showed the zirconia film to be thin. IR analysis shows that some tert-butoxy groups remain in the deposited and pyrolyzed film.

Further experiments with zirconium tetra(tert-butoxide) employing the following polymers and resins were performed with good results: polyimide Kapton®, polylactide-co-glycolate (PLGA), poly-3-hydroxybutyrate-co-valerate (PHBCV), Goretex, and Aramide. It is to be expected that similar treatment of other polymers will yield similar results.

### Example 2

### Reaction of Phosphonic Acid with Activated Polymers

Activated polymers produced in Example 1 were placed in a 0.1 mM solution of octadecylphosphonic acid (ODPA) in THF for 1 hour, giving phosphonate-bound polymer surfaces (with reference to FIG. 2, where M=Zr, and R==tert-butyl). Phosphonate-derivatized surfaces are effective at binding bio- or other classes of molecules.

### Example 3

### Metallization of Activated Polymers

Activated polymers produced in Example 1 were treated with an aqueous solution of a copper salt, which was absorbed onto the zirconium oxide adhesion layer. Treatment with either sodium borohydride or an amine borane gave a copper-coated polymer (with reference to FIG. 3, where M=Zr, and R=tert-butyl). Electron dispersive X-ray based analysis showed the presence of both copper and zirconium.

### Example 2a

### Reaction of Carboxylic Acid with Activated Polymers

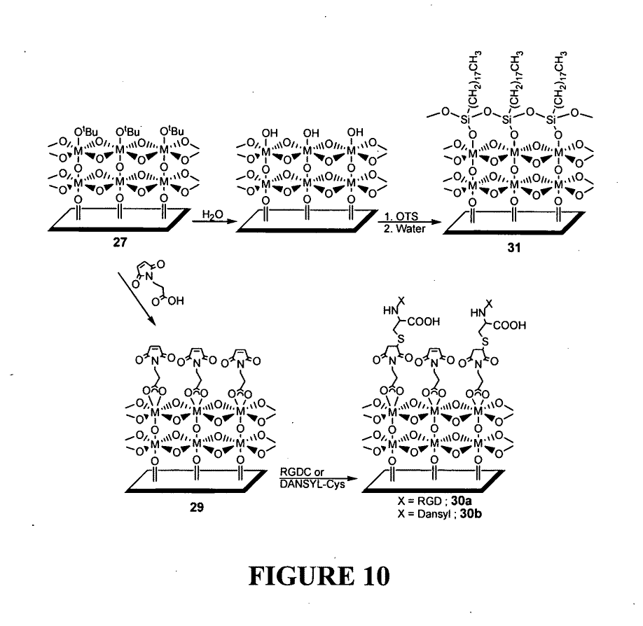

Activated PLGA polymer produced in Example 1 was placed in a 0.1 mM solution of maleimidopropionic acid in ethanol for 30 min, giving the maleimidocarboxlyate-bound polymer surface (with reference to FIG. 10, where M=Zr) to give 29. This derivatized surface is effective at binding bio- or other classes of molecules (30a and 30b).

### Example 3

### Metallization of Activated Polymers

Activated polymers of polyimides, aramides and Goretex composites produced as in Example 1 were treated with an aqueous solution of a copper salt, which was absorbed onto the zirconium oxide adhesion layer. Treatment with either sodium borohydride or an amine borane gave a copper-coated polymer (with reference to FIG. 3, where M=Zr, and R=tert-butyl). Electron dispersive X-ray based analysis showed the presence of both copper and zirconium.

Similarly, silver nitrate was used to deposit silver metal onto activated PET. It is to be expected that similar treatment of other polymers will yield similar results, as will the use of other metal salts using similar reducing agents.

### Example 3a

### Electroless Plating of Copper

A sample of Kapton treated first with the zirconium based adhesion layer, then copper sulfate, and then diethylamineborane as described in Example 3 was placed in a copper plating bath at 60° C. under nitrogen. The bath consisted of 0.1 M trisodium citrate dihydrate, 1.2 M ethylenediamine, 0.1 M copper sulfate hydrate, 0.03 M ferrous sulfate hydrate, 6.4×10⁻⁴ M 2,2-dipyridine, 1.2 M NaCl, and sufficient sulfuric acid to give pH=6. A small amount of PEG 200 (2.5 mg) was added to a 50 ml bath.

### Experiments

### Experiment 1

Details of the techniques and materials used herein are contained in the Experimental Section.

Formation of Metal Oxide/Alkoxide Adhesion Layers on Polymer Surfaces

Surface derivatization of solid polyethylene terephthalate (PET) and polyetheretherketone (PEEK) (shown below)

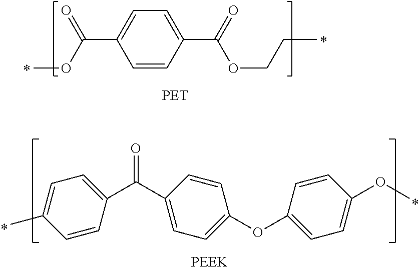

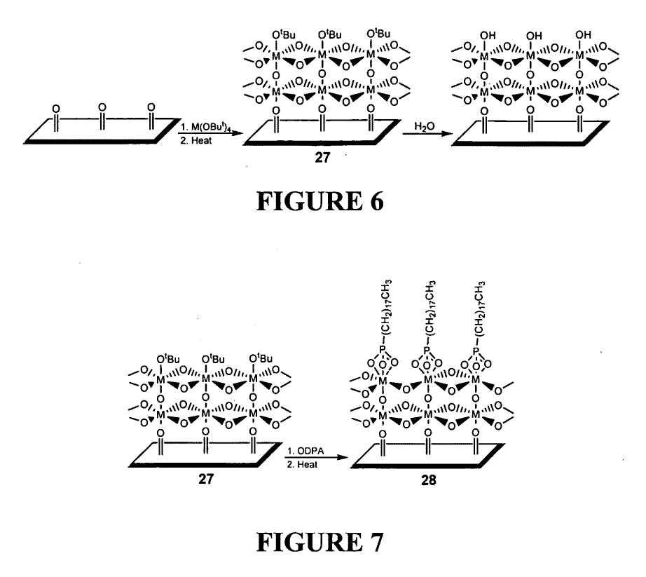

proceeded as follows. Polymer films (0.5 mm thick) were treated with vapor of zirconium tetra(tert-butoxide) (1) or titanium tetra(tert-butoxide) (2) and were then heated gently to 75° C. After heating, samples were sonicated for 1 min in dry THF (for PET) or acetonitrile (for PEEK). IR spectra of polymer surface-bound alkoxide/oxide adhesion layers (27) showed ν_{C-H}=2976 cm⁻¹, indicative of tert-butoxide groups, which initially gave a static water contact angle of 90° and which decreased to 35° by hydrolysis of tert-butoxy ligands when the samples were exposed to water overnight (FIG. 6).

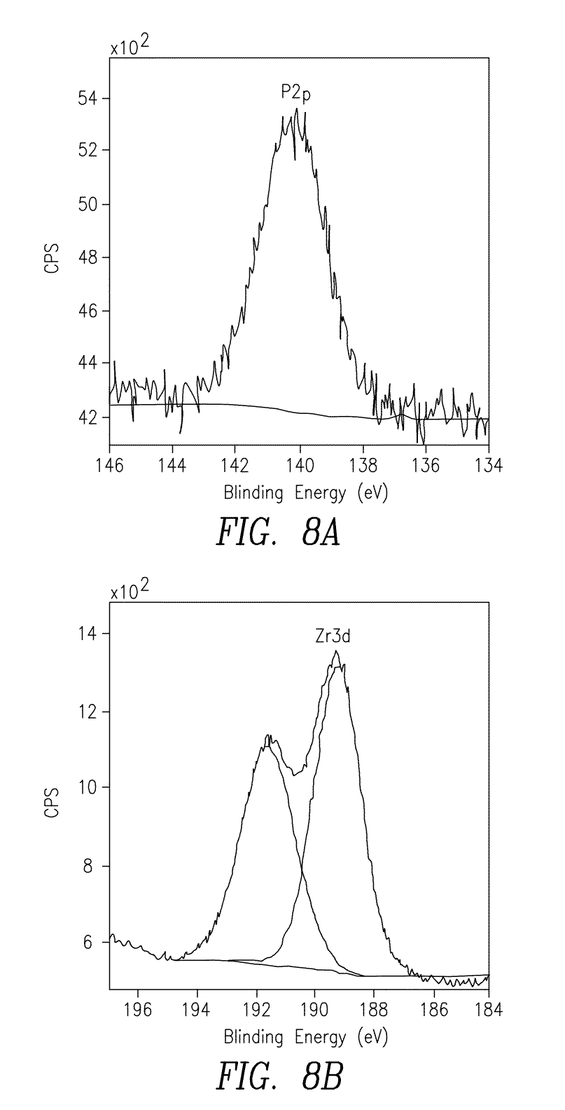

Now referring to FIG. 7, the adhesion layer 27-coated polymer films were treated with octadecylphosphonic acid (ODPA) via the tethering-by-aggregation-and-growth (T-BAG) method (see, Hanson, E.; Schwartz, J.; Nickel, B.; Koch, N.; Danisman, M. F. Bonding Self-Assembled, Compact Organophosphonate Monolayers to the Native Oxide Surface of Silicon. J. Am. Chem. Soc. 2003, 125, 16074-16080 (p. 16076, col. 1 lines 21-40), incorporated herein by reference) to yield surface 28, which had a water contact angle of 95° (Scheme 5-2). IR analysis of 28 showed peaks in the aliphatic region (ν_{CH2,asym}=2920 cm′; ν_{CH2,sym}=2849 cm⁻¹) characteristic of disordered alkyl chains. See, Gawalt, E. S.; Koch, N.; Schwartz, J. Self-Assembly and Bonding of Alkanephosphonic Acids on the Native Oxide Surface of Titanium, Langmuir 2001, 17, 5736-5738 (p. 5736, col. 2 lines 1-23), incorporated herein by reference. After soaking in deionized water for 24 h and 30 min of sonication in ethanol, X-ray photoelectron spectroscopy of octadecylphosphonate-coated PET (28) showed characteristic Zr (3d) and P (2p) bands FIGS. 8A and 8B, which suggested an overall Zr:P ratio of ≧2:1. This ratio is consistent with a model in which the Zr adhesion layer deposits as a bilayer (27), and only the topmost layer reacts with the octadecylphosphonate (28).

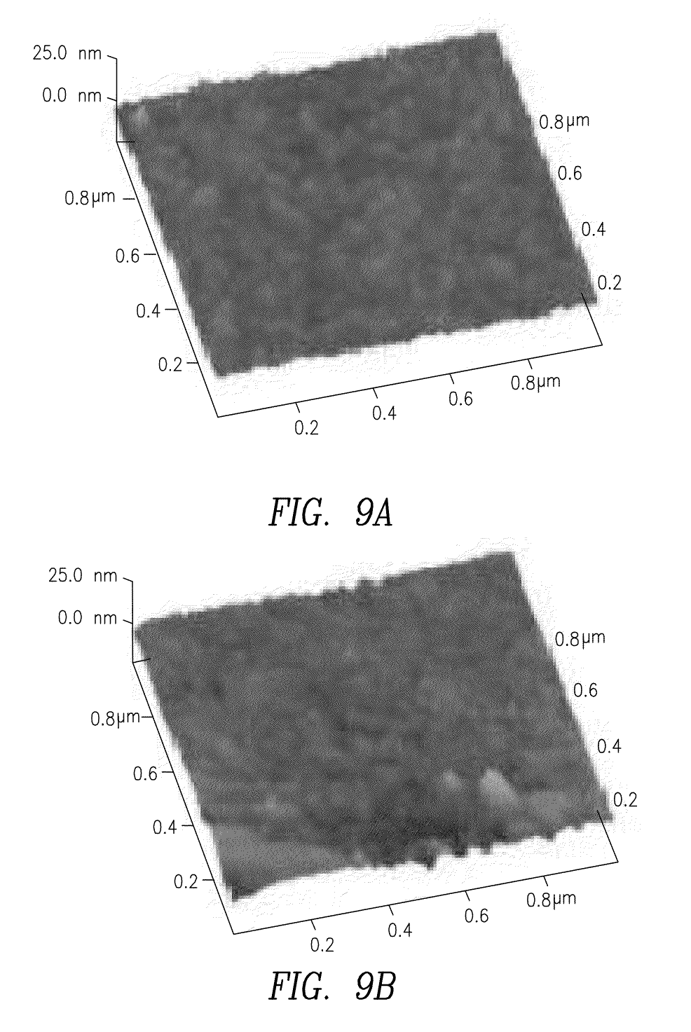

The coated PET was rigorously physically flexed and surface abraded with a Kimwipe®. Now referring to FIGS. 9A and 9B, AFM analysis of 28 showed a film depth of 3-4 nm, which is a reasonable height for the adhesion layer and ODPA; ODPA forms a ca. 2 nm thick film, which suggests a thickness of 1-2 nm for 27, and is consistent with XPS results.

The relationship between deposition and heating times of 1 and adhesion layer thickness was probed via quartz crystal microgravimetry (QCM). A silicon QCM crystal was placed in the deposition chamber with samples of PET and PEEK. The change in crystal frequency after deposition and heating was related via the Sauerbrey equation (3) to the mass of the adhesion layer that had been deposited on the crystal. Here, μ_{q} is the shear modulus of quartz (2.947×10¹¹ g/cm·s²) and p_{q} is the density of quartz (2.648 g/cm³).

$$Δ m = \frac{- Δ f \sqrt{{μ}_{q} {ρ}_{q}}}{4 {f}_{0}^{2}} \qquad ( 3 )$$

It was observed that longer deposition and heating times (1 hr) produced a ca. 8 nm surface layer, while shorter exposures (5 min) generated a ca. 1 nm layer (ca. 2 monolayers). Layer thicknesses were estimated assuming that the adhesion layer packs similarly to zirconia. To that end, the thickness was calculated as the quotient of the measured aerial surface density of 27 on the QCM crystal (in ng/cm²) and the known density of zirconia (5.89×10⁹ ng/cm³) (Table 1).

TABLE 1
Effect of deposition and heating time on adhesion layer thickness.

| Deposit Time | Heat Time | Δf/Δm | Thickness | |
|---|---|---|---|---|
| (min) | (min) | (Hz)/(ng/cm²) | (nm) | Monolayers |
| 5 | 10 | 289 ± 14/594 | 1 | 2 |
| 10 | 10 | 587 ± 11/1206 | 2 | 4 |
| 60 | 60 | 2295 ± 17/4717 | 8 | 16 |

Binding Organics to Polymers via the Adhesion Layer

Deposition of an adhesion layer 27 onto the surfaces of PET and PEEK activates them towards reaction with carboxylic acids, phosphonic acids, and silanes; this allows exquisite control of their surface wetting properties and enables the attachment of RGD or other cell-adhesive molecules in high yield. Now referring to FIG. 10, to efficiently tether RGD, polymers coated with 27 were immediately placed in a dry solution of 3-maleimidopropionic acid in acetonitrile to give 29, which is active for Michael addition of RGDC.

Silanes bound efficiently to PEEK and PET surfaces were derivatized with 27. The 27-coated polymer films were soaked for 1 hr in deionized water (to hydrolyze any remaining tert-butoxy ligands), blown dry, and soaked for 1 hr in a 0.1 mM solution of octadecyltrichlorosilane (OTS) in acetonitrile. The deposited silane films were crosslinked by soaking in deionized water for 5 min, after which the samples were cleaned by sonication in ethanol for 15 min, to give 31. Infrared spectra showed (ν_{CH2,asym}=2918 cm⁻¹; ν_{CH2,sym}=2848 cm⁻¹) indicative of disordered alkyl chains, and contact angle analysis indicated an increase in surface hydrophobicity (Θ=95° vs. 80° (PET) and 60° (PEEK)).

The relationship between the thickness of the adhesion layer 27 and the amount of ODPA or OTS bound to a sample surface was probed via QCM. Layers of the adhesion layer 27 with 2-16 monolayers were deposited on silicon-coated QCM crystals. The layers were hydrolyzed and then reacted with ODPA or OTS as described previously. The crystals were rinsed vigorously with methanol, and their frequencies were noted. Interestingly, the coverage (corrected for crystal surface roughness; R_{f}=1.3) (See, Carolus, M. D.; Bernasek, S. L.; Schwartz, J. Measuring the Surface Roughness of Sputtered Coatings by Microgravity. Langmuir 2005, 21, 4236-4239 (p. 4237, col. 1 lines 29-57), incorporated herein by reference) of OTS or ODPA appears to be independent of the thickness of 27 (Table 2). This indicates that only the topmost layer of 27 is reactive toward organic functionalization.

TABLE 2
QCM quantification of OTS and ODPA coverage on adhesion layer 27

| 27 Thickness | ΔF OTS | OTS Coverage | ΔF ODPA | ODPA Coverage |
|---|---|---|---|---|
| (nm) | (Hz) | (nmol/cm²) | (Hz) | (nmol/cm²) |
| 1 | 303 ± 3 | 1.33 | 185 ± 5 | 0.94 |
| 2 | 306 ± 5 | 1.35 | 192 ± 4 | 0.98 |
| 8 | 300 ± 7 | 1.31 | 190 ± 2 | 0.97 |

Surface loading of organics bound to PET and PEEK were determined through 30a by reaction with DANSYL-cys to give 30b. The fluorescently-labeled polymers were monitored by fluorescence spectroscopy using techniques as described in Dennes, T. J.; Hunt, G. C.; Schwarzbauer, J. E.; Schwartz, J. High-Yield Activation of Scaffold Polymer Surfaces to Attach Cell Adhesion Molecules. J. Am. Chem. Soc. 2007, 129, 93-97 (p. 94, below Schemes 1 & 2, col. 2 lines 25-34; p. 95, below Scheme 3, col. 1 lines 1-20; p. 96, below FIG. 1, col. 2 lines 1-24; p. 97, col. 1 lines 1-15 & col. 2 lines 1-3) and Danahy, M. P.; Avaltroni, M. J.; Midwood, K. S.; Schwarzbauer, J. E.; Schwartz, J. Self-assembled Monolayers of α,ω-Diphosphonic Acids on Ti Enable Complete or Spatially Controlled Surface Derivatization. Langmuir 2004, 20, 5333-5337 (p. 5335, below Scheme 3, col. 1 lines 1-15), incorporated herein by reference, to measure the stability of 30b in aqueous conditions and to quantify the amount of material bound to the polymer surface. After the initial removal of synthetic residues, PET and PEEK coated with 30b showed no desorption of fluorescent material over 7 days. When 30b was cleaved from the polymer surface by treatment with aqueous solution at pH 12.5, 90 pmol/cm² was measured on both PET and PEEK surfaces, indicating sub-monolayer coverage of the adhesion layer 27.

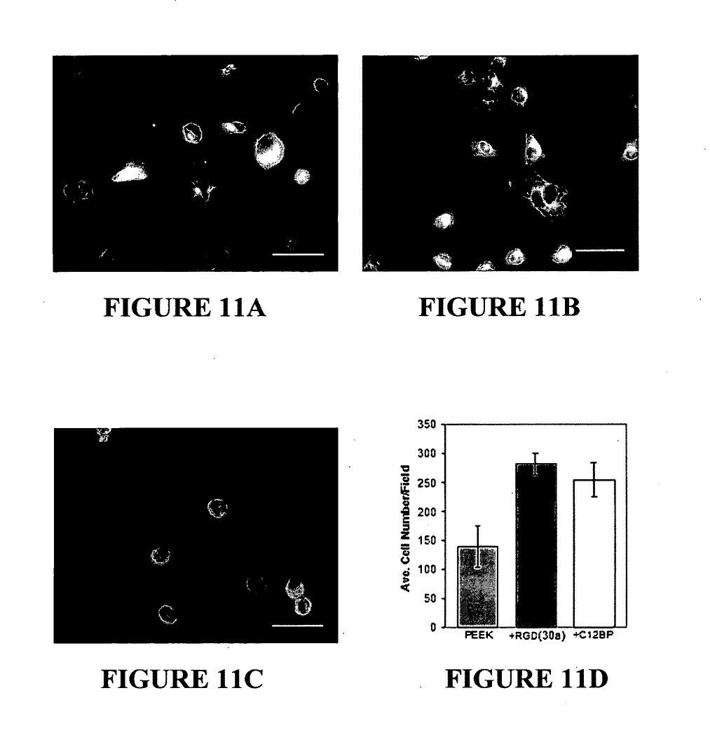

Now referring to FIGS. 11A-11D, in vitro experiments were conducted with osteoblast cells to evaluate osteoblast attachment on derivatized PEEK, 30a, and PEEK-C12BP, which was prepared by deposition of 1,12-dodecylbisphosphonic acid on 27 via the T-BAG method referenced hereinabove. FIG. 11A depicts cells on RGD-modified PEEK (30a), FIG. 11B depicts C12BP-modified PEEK, and FIG. 11C depicts PEEK control surfaces after 3 h, fixed and stained with anti-vinculin antibodies and fluorescein-conjugated secondary antibodies. Scale bars are 50 μm. FIG. 11D indicates then number of cells per 10× microscope field counted for untreated PEEK, RGD-derivatized, and C12BP-derivatized PEEK. Average values from at least three fields are shown with error bars representing ±1 standard deviation. Both 30a and PEEK-C12BP showed increased osteoblast adhesion after 3 h versus the PEEK control (p=2.3×10⁻⁴ and 6.3×10⁻⁴, respectively); 30a supported significantly greater osteoblast spreading when compared to a PEEK control (FIG. 11C).

Adhesion Layer Interfacial Shear Strength Testing

Evaluating interfacial resistance to shear force can help determine a coating's mechanical stability and its utility in implant applications. The current standard in orthopedic implant technology utilizes an hydroxyapatite coating with an interfacial shear strength of ca. 10 MPa. See, Silverman, B. M.; Wieghaus, K. A.; Schwartz, J. Comparative Properties of Siloxane vs. Phosphonate Monolayers on a Key Titanium Alloy. Langmuir 2005, 21, 225-228 (p. 227, below Table 1, col. 1 lines 1-16), and Schwartz, J.; Avaltroni, M.; Danahy, M. Cell attachment and spreading on metal implant materials. Materials Science & Engineering C 2002, 23, 395-400 (p. 398, col. 2 lines 1-9; p. 399, col. 1 lines 1-6), incorporated herein by reference. To determine the unoptimized interfacial shear strength of adhesion layer 27 on PEEK, a modified version of a previously reported (Silverman et al, Langmuir 2005, 21, 225-228) shear strength test was developed. Coupons of PEEK were coated with adhesion layer 27 and epoxy-glued to Ti-6A1-4V coupons. Shear force was exerted parallel to the interface on the glued coupons until a breaking point was achieved. These tests measured interfacial shear strength of 7.8±0.2 MPa before failure for adhesion layer 27, compared with 3.0±0.2 MPa for control PEEK.

Thus nanoscale metal oxide/alkoxide adhesion layers 27 generated on the surfaces of PEEK and PET are effective for activation of those polymers for further organic chemical transformation. Silanes, carboxylic acids, and phosphonic acids can be easily attached to PET and PEEK through adhesion layer 27, which allows comprehensive control of their surface wetting properties. This approach was illustrated by tethering cell attractive peptide RGD to the surface of PEEK in high yield (90 pmol/cm² or 20% surface coverage). RGD attachment to PEEK films via adhesion layer 27 proved effective to increase osteoblast adhesion and spreading on that surface; in addition, PEEK surfaces derivatized with C12BP were shown to increase cell adhesion. Since this activation process involves metal complex coordination to surface groups, it is broadly applicable to other polymers that contain such groups, including polyamides, polyurethanes, polyimides, and poly-thiophenes.

### Experimental Section

General.

All reagents were obtained from Aldrich and used as received unless otherwise noted. IR spectra were collected using a Midac Model 2510 spectrometer equipped with a Surface Optics Corp. specular reflectance attachment. Fluorimetry measurements used a Photon Technology International Fluorescence Spectrometer. AFM analysis of films was done using a Digital Instruments Multimode Nanoscope Ma SPM equipped with silicon tips (Nanodevices Metrology Probes; resonant frequency, 300 kHz; spring constant, 40 N/m) in tapping mode. Quartz crystal microbalance (QCM) measurements were made using an International Crystal Manufacturing standard (clock) oscillator, model 35360, and 10 MHz, AT-cut quartz crystals (ICM) equipped with SiO₂/Si-coated (1000 Å Si/100 Å Cr/1000 Å Au undercoat) electrodes. Curve fitting of core-level XPS peaks was done using CasaXPS software with a Gaussian-Lorentzian product function and non-linear Shirley background subtraction. Standard atomic photoionization cross-section values from the SPECS database were used for quantitative estimations of surface compositions. Dubey, M.; Gouzman, I.; Bernasek, S. L.; Schwartz, J. Characterization of Self-Assembled Organic Films Using Differential Charging in X-Ray Photoelectron Spectroscopy. Langmuir 2006, 23, 4649-4653 (p. 4650, col. 1 lines 41-55).

Metal Oxide/Alkoxide Adhesion Layers.

Coupons of PET, PEEK, and Kapton® polyimide film (Goodfellow) and a QCM crystal were placed in a deposition chamber that was equipped with two stopcocks for exposure either to vacuum or to vapor of zirconium tetra(tert-butoxide) (1) or titanium tetra(tert-butoxide) (2). The chamber was evacuated to 10⁻³ torr for 30 minutes, and polymer films were exposed to vapor of 1 or 2 (with external evacuation) for 30 seconds followed by 5 min exposure without external evacuation. At this time, the stopcock of the metal alkoxide was closed, heating tape was applied, and the samples were heated to 75° C. for 5 min, then allowed to cool to room temperature. The chamber was then evacuated for 30 min at 10⁻³ ton to ensure removal of excess 1 or 2 and to give surface activated polymers. The QCM crystal was rinsed with THF and methanol; its measured change in frequency indicated the amount of alkoxide complex that had been deposited. The above procedure yields an adhesion layer of ca. 1 nm, or two monolayers. If exposure and heating times are increased, thicker layers can be achieved (Table 1).

Formation of Surface Metal-Carboxylate Complexes.

Polymer surfaces activated with the metal oxide/alkoxide adhesion layer were placed in a dry solution of either 3-maleimidopropionic acid (0.1 mM) in acetonitrile for 1 hr to generate maleimido-derivatized surfaces, or 0.1 mM solutions of DANSYL-cys or fluorescein for 1 hr to generate fluorophore-derivatized surfaces.

Silanization of Polymer Surfaces.

After deposition of the metal oxide/alkoxide adhesion layer, polymers were soaked in deionized water for 1 hr to hydrolyze all remaining tert-butoxide ligands. The hydrolyzed surfaces were dried in vacuo and were then soaked for 1 hr in a dry 0.1 mM solution of 3-aminopropyltriethoxysilane or octadecyltrichlorosilane in acetonitrile. The silane films were soaked in a 75/25 (v/v) solution of acetonitrile/water for 15 min for crosslinking, and were then sonicated first in acetonitrile for 15 min, then in ethanol for 15 min.

Formation of Phosphonate Monolayers on Polymer Surfaces.

Polymer surfaces derivatized with adhesion layer 27 were hydrolyzed in water overnight, sonicated in ethanol for 5 min, and functionalized via the T-BAG method as previously described. Briefly, polymer samples were suspended in a 0.1 mM solution of a phosphonic acid (11-hydroxyundecyl phosphonic acid or octadecylphosphonic acid in THF, or 1,12-dodecylbisphosphonic acid in 95/5 (v/v) THF/methanol). The solvent was allowed to evaporate over 3-5 hrs, and samples were then baked at 120° C. (below the T_{g}) for 24 hrs. Samples were sonicated in ethanol for 15 min, yielding phosphonate monolayers. The use of THF as a solvent with PEEK is to be avoided; methanol is used instead.

Determination of Hydrolytic Stability and Surface Loading.

DANSYLated polymer films were immersed in pH 7.5 aqueous solution for 3 days and were monitored via fluorescence spectroscopy for loss of DANSYL from the polymer surfaces. Surface loading was determined via cleavage of the remaining DANSYL molecules in aqueous solution (pH 12.5) for 3 hrs.

Quartz Crystal Microgravimetry Crystal Roughness Factor (Rf) Determination.

Surface roughness was measured using a modified Brunauer-Emmett-Teller (BET) experiment. See, Carolus, M. D.; Bernasek, S. L.; Schwartz, J. Measuring the Surface Roughness of Sputtered Coatings by Microgravity. Langmuir 2005, 21, 4236-4239 (p. 4237, col. 1 lines 29-57), incorporated herein by reference. An ICM oscillator drove silicon QCM crystals whose resonant frequencies were monitored using a Hewlett Packard 5200 series frequency counter. Each QCM crystal was rinsed with methanol, blown dry in a stream of N₂, and mounted in a vacuum chamber equipped with ports for electrical wiring. The pressure inside the chamber was reduced to less than 1 torr and the frequency of the QCM crystal was allowed to stabilize. The chamber was next isolated from active vacuum, and opened to a vial containing tetramethylsilane (TMS), which was held in a water bath at room temperature. The vacuum chamber was filled with TMS (Pᵥₐₚ=630 torr), reevacuated, and again filled with TMS while frequency readings were recorded in 10 torr increments. An adsorption isotherm was obtained by plotting the frequency of the crystal from 0-630 torr of TMS. The roughness factor was calculated as follows: A plot was made of χ/Δf(1−χ) versus χ, where χ is the partial pressure of TMS, for 0.05<χ<0.35. A linear fit of this plot gave a slope and intercept, which was used to obtain a dimensionless constant, C (4), and allowed calculation of the frequency change at monolayer coverage (fₘ, (5)). The Sauerbrey equation (3) allowed calculation of the mass of probe molecules adsorbed at monolayer coverage, which could be extrapolated to an area for monolayer coverage of TMS, assuming the molecular “footprint” of TMS to be 40 Å². The roughness factor was calculated as the quotient of the monolayer derived area and the nominal area of the QCM crystal.

C=1+slope/intercept (4)

fₘ=1/[C(intercept)] (5)

In Vitro Cell Response.

Osteoblast response to PEEK surfaces was evaluated in vitro. Osteoblasts maintained in DMEM with glutamine, Penn Strep, G418, and 10% calf serum (Hyclone) were removed from TCPS plates using 0.1 mg/mL trypsin LE express (Invitrogen) and were prepared as previously described. See, Danahy, M. P.; Avaltroni, M. J.; Midwood, K. S.; Schwarzbauer, J. E.; Schwartz, J. Self-assembled Monolayers of α,ω-Diphosphonic Acids on Ti Enable Complete or Spatially Controlled Surface Derivatization. Langmuir 2004, 20, 5333-5337 (p. 5335, below Scheme 3, col. 1 lines 16-33). Cells (1.0×10⁵ cells/mL in serum-free media) were added to 24-well tissue culture plates containing PEEK samples and incubated at 34° C. After 90 min, medium was replaced with fresh, serum-free DMEM. At 3 h cells were fixed, permeabilized, and stained with anti-vinculin antibody (Sigma) followed by fluorescein goat anti-mouse secondary antibody. Images were obtained as described previously. See, Midwood, K. S.; Schwarzbauer, J. Tenascin-C Modulates Matrix Contraction via Focal Adhesion Kinase- and Rho-mediated Signaling Pathways. Mol. Biol. Cell 2002, 13, 3601 (p. 3602, col. 2 lines 5-57). Cell adhesion was quantified by counting the number of attached cells in at least 3 microscope fields.

Shear Strength Testing.

PEEK coupons (Goodfellow) were cut to be 1.125″×0.5″. These PEEK coupons were sanded with 220- and 400-grit SiC paper to a smooth finish, sonicated in EtOH for 15 min, and surface functionalized with adhesion layer 27. The adhesion layer-coated surfaces were hydrolyzed in water for 5 min, sonicated in EtOH for 15 min, and joined to clean Ti-6A1-4V coupons using a 1.5 cm² piece of Cytec Fiberite FM 1000 epoxy, which was placed between the coupons in a vise. The samples were heat-cured by ramping the oven temperature from 25° C. to 170° C. at 2 degrees per min, and holding the temperature at 170° C. for 90 min. The joined coupons were placed in a stainless steel holder, which was placed in an Instron Model 1331 load testing machine, and the samples were pulled apart at 100 μm/sec while a computer interface recorded the point of maximum shear stress, when failure occurred.

### Experiment 2

### Patterned Zirconium Oxide/Alkoxide Adhesion Layers on PET and PEEK

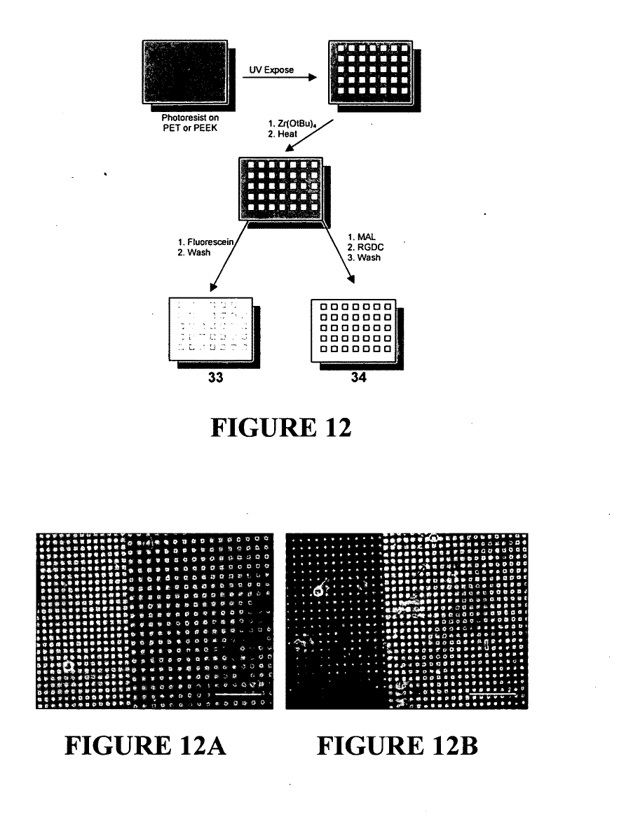

The deposition of metal oxide/alkoxide adhesions layers on polymers is also amenable to spatial control via photolithography. The initial chemical vapor deposition of a metal alkoxide precursor allows precise control over spatial substrate exposure. Now referring to FIG. 12, to demonstrate this, samples of PET and PEEK were patterned with photoresist as described in detail below in the below Experimental Section, and treated with vapor of zirconium tetra(tert-butoxide) (1). The samples were heated to generate patterned areas of the zirconium oxide/alkoxide adhesion layer 27 and reacted with 3-maleimidopropionic acid (to give a surface active for RGDC coupling) or fluorescein (to generate samples for fluorescence microscope imaging). Samples were sonicated in acetone to remove the remaining photoresist, giving 33 and 34. Now referring to FIGS. 12A and 12B, when 33 was imaged with a fluorescence microscope, patterned features as small as 2×2 μm were observed; this is likely sufficient resolution to allow control of cell shape.

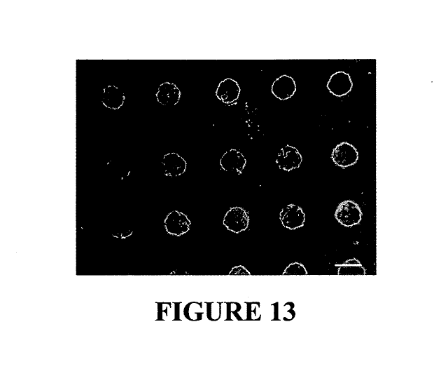

A reaction scheme was devised that would allow two species to be patterned at the surfaces of PET and PEEK. Clean samples of PET and PEEK were exposed to vapor of 1 and heated to give comprehensive coverage of adhesion layer 27, which was hydrolyzed in water for 5 min. The polymers were next patterned photolithographically and were again exposed to vapor of 1 and heated to give patterned areas of adhesion layer 27, which were reacted immediately with 3-maleimidopropionic acid (to generate RGDC binding sites) or fluorescein (for fluorescence microscope imaging). Next, samples were sonicated in acetone to remove remaining photoresist, derivatized with HUPA via the T-BAG method, and reacted with PEG-SS (to deactivate the background for cell adhesion) or 5(6)-carboxy-X-rhodamine N-succinimidyl ester (for fluorescence microscope imaging). Now referring to FIG. 13, fluorescently-labeled samples indicated that the reaction scheme was successful, with fluorescein clearly bound in active areas and rhodamine bound in background areas.

### Evaluation of Cell Response to Patterned Substrates

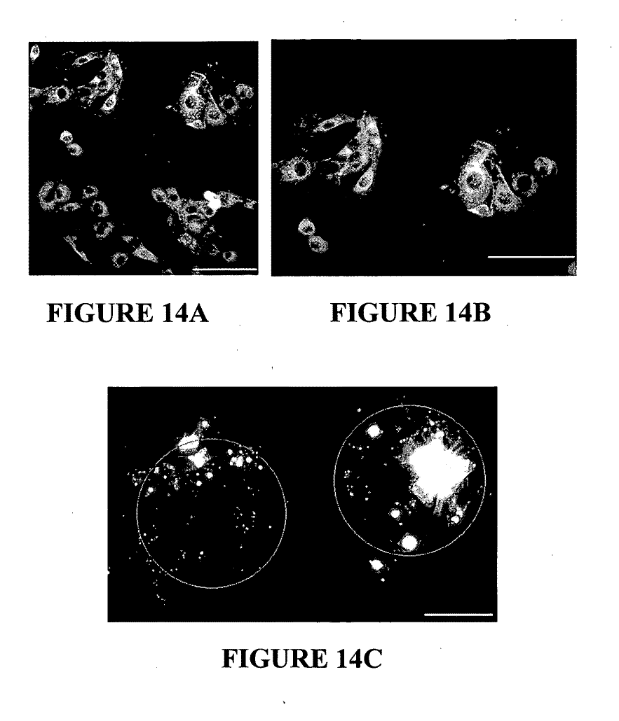

The response of NIH3T3 cells to 32-derivatized Nylon 6/6 and 34-derivatized PET was observed in vitro. Now referring to FIGS. 14A-14C, cells adhered preferentially to the RGD-patterned areas of 32 within 3 h, and on staining for vinculin, focal adhesions were visualized. Cells seeded on RGD islands on Nylon 6/6 (FIGS. 14A and 14B) and PET (FIG. 14C) are depicted. Cells were stained for vinculin-containing focal adhesions (FIGS. 14A and 14B) and labeled with Cell Tracker Green (Molecular Probes, Inc). Circles indicate pattern boundaries in FIG. 14C. Cells clustered inside square RGD “islands” and spread to fill them. On 34, cells were labeled with Cell Tracker Green (Molecular Probes, Inc.) and allowed to attach and spread for 3 h. Cells preferentially attached and spread inside circular RGD islands.

Thus, spatially-controlled derivatization of Nylon 6/6, PET, and PEEK was made possible by the coupling of traditional photolithography with the chemical vapor deposition of zirconium alkoxides. Fluorescence microscopy showed the conformance of the chemistry to the predetermined patterns, and in vitro cell response on the patterned polymers was achieved, allowing spatial control of cell adhesion and spreading on the polymer substrates. Doubly-patterned substrates are believed to improve the ability of patterned polymeric surfaces to holistically control cell attachment and morphology.

### Experimental Section

General.

All chemicals were obtained from Aldrich and used as received unless otherwise noted. Acetonitrile and THF were dried over CaH₂ and KOH, respectively, and were distilled prior to use. Fluorescence microscope images were obtained using a Nikon Optiphot-2 microscope (Garden City, N.Y.), and images were captured using a Photometrics Coolsnap camera (Tucson, Ariz.) and analyzed using IP lab software.

Photolithography.

Films of nylon 6/6, PET, Kapton® polyimide film, and PEEK (Goodfellow) were sonicated in ethanol for 15 minutes and blown dry in a stream of N₂ prior to use. For spatial control of surface functionalization, polymer films were spin-coated with 2 drops AZ® 5214-E photoresist at 4000 rpm for 30 s. The samples were annealed at 95° C. for 45 s, and were exposed to UV (365 nm) at 950 mW/cm² for 5 min. Samples were developed in a 50/50 (v/v) solution of AZ® 312 MIF developer and water for 1 minute, rinsed in deionized water, blown dry in a stream of N₂, and evacuated at 10⁻³ torr for 1 hr.

Spatially-Controlled Formation of Zr-Amidate.

Photopatterned nylon 6/6 was placed in a deposition chamber that was equipped with two stopcocks for exposure either to vacuum or to vapor of 1. The chamber was evacuated to 10⁻³ torr for 30 minutes, and polymer films were exposed to vapor of 1 (with external evacuation) for 30 seconds followed by 5 min exposure without external evacuation. This cycle was repeated twice, and the chamber was then evacuated for 30 min at 10⁻³ torr to ensure removal of excess 1, and gave surface amidate complexes.

Spatially-Controlled Formation of Metal Oxide/Alkoxide Adhesion Layers.

Photopatterned PET, PEEK, and Kapton® (a registered trademark of DuPont) polyimide film (obtained from Goodfellow) were placed in a deposition chamber that was equipped with two stopcocks for exposure either to vacuum or to vapor of 1. The chamber was evacuated to 10⁻³ torr for 30 minutes, and polymer films were exposed to vapor of 1 (with external evacuation) for 30 seconds followed by 5 min exposure without external evacuation. At this time, the stopcock for the metal alkoxide was closed, a heating tape was applied, and the samples were heated to 75° C. for 5 min, and allowed to cool to room temperature. The chamber was then evacuated for 30 min at 10⁻³ torr to ensure removal of excess 1, and to give surface activated polymers. The above procedure yields an adhesion layer of ca. 1 nm. If exposure and heating times are increased, thicker layers can be achieved.

Fluorophore Derivatization.

Patterned samples were immersed in a dry, 0.1 mM solution of fluorescein in acetonitrile for 1 hr, removed, and rinsed/sonicated in acetone to remove remaining photoresist. Sonication for 15 min in ethanol followed by drying in a stream of N₂ gave fluorescein patterned polymers that were imaged with a fluorescence microscope.

RGD Derivatization.

Patterned samples were immersed in a dry 0.1 mM solution of 3-maleimidopropionic acid for 1 hr and sonicated in acetone to remove remaining photoresist. After a 15 min sonication in ethanol, samples were placed in a 0.1 mM aqueous solution of RGDC (American Peptide) for 24 hrs, which was adjusted to pH 6.5 using aqueous NaOH. Samples were soaked in deionized water for 30 min, blown dry in a stream of N₂, and stored for tissue culture studies.

Doubly-Patterned RGD and PEG Derivatization on Nylon 6/6.

Zr-complex patterned samples were immersed in a dry 0.1 mM solution of 3-maleimidopropionic acid for 1 hr and sonicated in acetone to remove remaining photoresist. After a 15 min sonication in ethanol, samples were blown dry with N₂ and placed in a deposition chamber that was equipped with two stopcocks for exposure either to vacuum or to vapor of 1. The chamber was evacuated to 10⁻³ torr for 30 minutes, and polymer films were exposed to vapor of 1 (with external evacuation) for 30 seconds followed by 5 min exposure without external evacuation. This cycle was repeated twice, and the chamber was then evacuated for 30 min at 10⁻³ torr to ensure removal of excess 1, and gave surface amidate complexes that backfilled the previously blank areas. Samples were placed in a dry 0.1 mM solution of 11-hydroxyundecylphosphonic acid in THF for 1 hr, sonicated in ethanol for 15 min, and blown dry in a stream of N₂. Samples were next placed in a dry 0.1 mM solution of polyethylene glycol succinimidyl succinate (5000 MW, Laysan Inc.) in acetonitrile for 36 hrs. After a 15 min ethanol sonication, samples were placed in a 0.1 mM aqueous solution of RGDC (American Peptide) for 24 hrs, which was adjusted to pH 6.5 using aqueous NaOH. Samples were soaked in deionized water for 30 min, blown dry in a stream of N₂, and stored for tissue culture studies.

Doubly-Patterned RGD and PEG Derivatization on PET and PEEK.

Clean polymer samples were placed in a deposition chamber that was equipped with two stopcocks for exposure either to vacuum or to vapor of 1. The chamber was evacuated to 10⁻³ torr for 30 minutes, and polymer films were exposed to vapor of 1 or 2 (with external evacuation) for 30 seconds followed by 5 min exposure without external evacuation. At this time, the stopcock for the metal alkoxide was closed, a heating tape was applied, and the samples were heated to 75° C. for 5 min, and then allowed to cool to room temperature. The chamber was then evacuated for 30 min at 10⁻³ torr to ensure removal of excess 1, and to give surface activated polymers. The polymers were sonicated for 1 min in dry THF, and subsequently hydrolyzed in water for 5 min. Samples were photolithographically patterned as previously described and were again placed in a deposition chamber that was equipped with two stopcocks for exposure either to vacuum or to vapor of 1 or 2. The chamber was evacuated to 10⁻³ torr for 30 minutes, and polymer films were exposed to vapor of 1 or 2 (with external evacuation) for 30 seconds followed by 5 min exposure without external evacuation. This cycle was repeated twice, and the chamber was then evacuated for 30 min at 10⁻³ torr to ensure removal of excess 1 to give surface metal complexes that were reacted immediately with 3-maleimidopropionic acid (0.1 mM solution in dry acetonitrile) for 1 hr. The remaining photoresist was washed away in acetone and a layer of 11-hydroxyundecylphosphonic acid was backfilled into the unreacted areas via the T-BAG method of Hansen et al. previously referenced hereinabove (J. Am. Chem. Soc. 2003, 125, 16074-16080). The samples were reacted with a 0.1 mM solution of polyethylene glycol succinimidyl succinate (PEG-SS, 5000 MW, Laysan) in dry acetonitrile for 36 hrs. The samples were sonicated in ethanol and reacted for 24 hrs in a 0.1 mM solution of RGDC (American Peptide), which had been adjusted to pH 6.5 with NaOH (aq). Doubly-patterned samples were soaked in deionixed water for 30 min, blown dry in a stream of N₂, and stored in a desiccator for later use.

Doubly-Patterned Fluorophore Derivatization.

Polymer samples were treated in the same way as described for doubly-patterned RGD and PEG, but fluorescein was used in lieu of 3-maleimidopropionic acid, and 5(6)-carboxy-X-rhodamine N-succinimidyl ester (Fluka) was used in lieu of PEG succinimidyl succinate. Samples were visualized with a fluorescence microscope to show correspondence with the expected pattern.

In Vitro Cell Performance.

Cell response to polymer surfaces was evaluated in vitro. NIH3T3 cells maintained in Dulbecco's Modified Eagle's Medium (DMEM) with 10% calf serum (Hyclone) were prepared for cell adhesion experiments as previously described (Danahy et al., Langmuir 2004, 20, 5333-5337). Cells (1×10⁵/mL in DMEM with 10% calf serum) were added to 24-well tissue culture dishes containing untreated or derivatized polymer surfaces. After 90 minutes, medium with non-adherent cells was removed and replaced with fresh DMEM. At 3 hr cells were fixed, permeabilized, and stained with anti-vinculin antibody (Sigma) followed by fluorescein goat anti mouse secondary antibody (for focal adhesions), and DAPI (for DNA). Images were obtained as described previously (Midwood et al., Mol. Biol. Cell 2002, 13, 3601-3613). Brightness and contrast of color levels were adjusted with IPLab software.

Control of Stem Cell Differentiation.

It has been shown that the shape of mesenchymal stem cells (MSC's) influences their differentiation. Chen, et al., Micropatterned surfaces for control of cell shape, position, and function, Biotechnol. Prog. 1998, 14, 356-363 (p. 357, col. 2 lines 36-67 and p.360, below FIG. 3, col. 1 lines 6-19); McBeath et al., Cell Shape, Cytoskeletal Tension, and RhoA Regulate Stem Sell Lineage Commitment. Develop. Cell 2004, 6, 483-495 (p. 485-486; p. 487, col. 2 lines 1-8; p. 488-489; p. 490, col. 2 lines 1-43), incorporated herein by reference. MSC's that were forced to spread maximally inside 100 μm² fibronectin squares (adsorbed on tissue-culture polystyrene) differentiated preferentially into osteoblasts, while MSC's constrained on circles of 10 μm diameter became adipocytes (FIG. 7-1). Since simple adsorption is not a favorable surface functionalization scheme for a device (see, Falconnet et al., Surface engineering approaches to micropattern surfaces for cell-based assays Biomaterials 2006, 27, 3044-3063, (p. 3045, col. 1 lines 17-28)), the new polymer surface-patterning schemes presented hereinabove present an alternative for improved control of cell shape on a polymeric tissue scaffold, enabling preferential development of different tissues at different sites in a scaffold.

### Experiment 3

### Polymer Metallization

As described hereinabove, the zirconium oxide/alkoxide adhesion layer 27 nucleates the growth of copper metal on and adhesion to PET and Kapton® polyimide film surfaces; this approach provides a basis for patterned metallization of polymer-based device substrates.

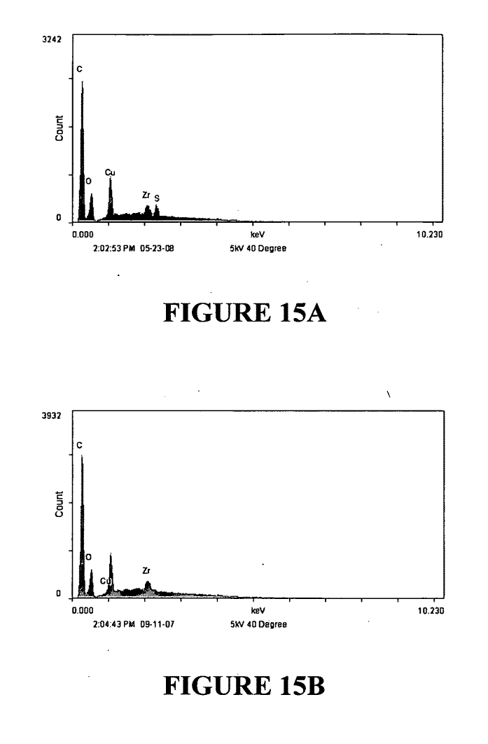

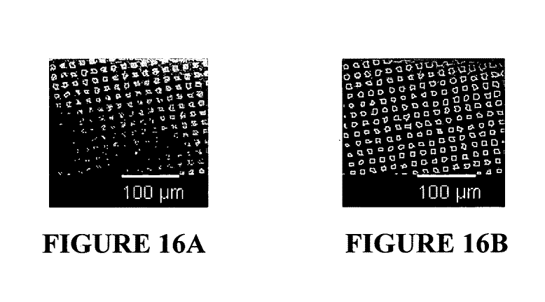

Adhesion layer 27 can serve as a matrix to enable polymer surface metallization. In a typical procedure Kapton® polyimide film was coated with a 5 nm thick layer of adhesion layer 27 and was then soaked in a 200 mM aqueous solution of CuSO₄. Samples were rinsed in deionized water, and EDX analysis confirmed the presence of Cu and S (FIG. 7-2). After subsequent (slow) reduction by dimethylamine borane (1 M, aqueous, 6 hrs, 50° C.), metallic copper was formed. Metallization was also done using adhesion layer 27 patterned on Kapton® polyimide film. The metallized surface was subjected to sonication in water and physical rubbing with a Q-tip, which was followed by EDX (FIGS. 15A and 15B). In this way it was shown that patterns of both Zr and Cu on the Kapton® polyimide film surface faithfully replicated the mask design (FIGS. 16A and 16B).

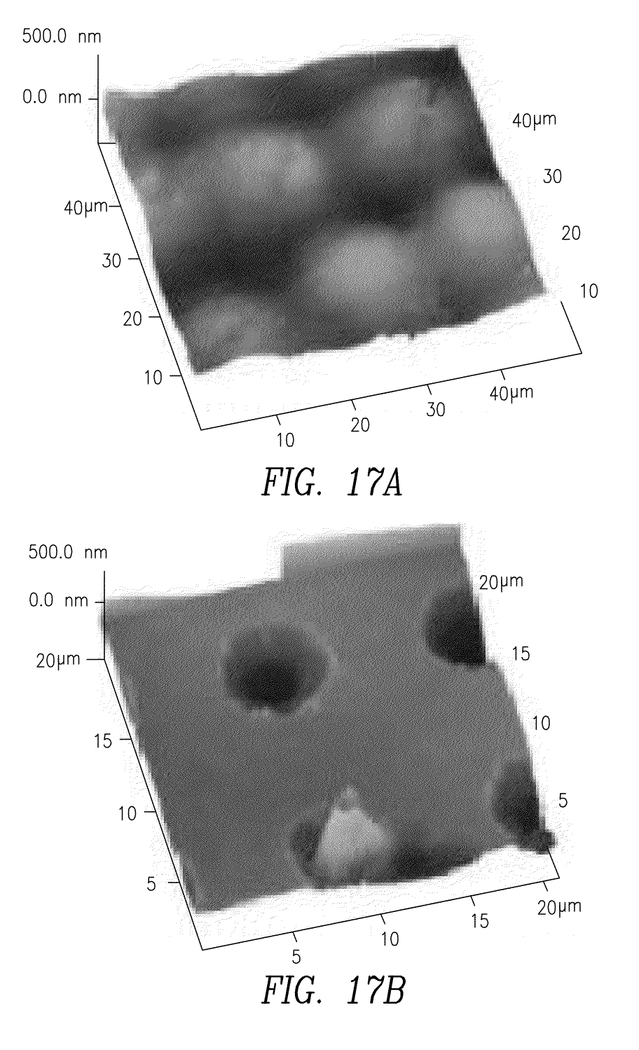

Now referring to FIGS. 17A and 17B, a corresponding pattern was also observed by AFM. The thickness of the generated copper “seed” was measured via AFM to be ca. 20 times thicker than the starting film of adhesion layer 27 (FIG. 17A); indicating that adhesion layer 27 nucleates the growth of CuSO₄ (observed by EDX, FIGS. 15A and 15B) at the polyimide surface. Interestingly, CuSO₄-treated Kapton® polyimide film was reduced rapidly using aqueous sodium borohydride, which also gave copper metal; here, AFM analysis shows the Cu pattern to be buried into the polymer surface in pits the tops of which in many cases were about 500 nm below the polymer surface (FIG. 17B). It is believed that the relatively faster borohydride reduction is sufficiently exothermic so that the polymer melts in the vicinity of the reduction reaction.

Because adhesion layer 27 is thin (ca. 5 nm), it is resistant to cracking by physically flexing the polymer, adhesion layer 27 is a suitable matrix for polymer metallization with copper. Copper “seed” layers can serve as nucleation sites for bulk copper growth by electroless deposition processes (Gu et al., Organic Solution Deposition of Copper Seed Layers onto Barrier Metals. Mat. Res. Soc. Symp. Proc. 2000, 612, D9.19.1-D9.19.6 (p. D9.19.2, lines 33-40; p. D9.19.5, lines 14-22)). In conjunction with photolithographic patterning, this further metallization of the polymer provides a means to prepare copper-based electrical circuitry on a variety of flexible substrates under simple laboratory conditions.

### Experimental Section

General.

All chemicals were obtained from Aldrich and used as received unless otherwise noted. Acetonitrile and THF were dried over CaH₂ and KOH, respectively, and were distilled prior to use. Fluorescence microscope images were obtained using a Nikon Optiphot-2 microscope (Garden City, N.Y.), and images were captured using a Photometrics Coolsnap camera (Tucson, Ariz.) and analyzed using IP lab software. AFM analysis of films used a Digital Instruments Multimode Nanoscope IIIa SPM equipped with silicon tips (Nanodevices Metrology Probes; resonant frequency, 300 kHz; spring constant, 40 N/m) in tapping mode.

Metallization of Kapton® Polyimide Film and PET.

Patterned or un-patterned copper metallization of the polymer surfaces was achieved by soaking an activated polymer surface in a 200 mM aqueous solution of CuSO₄ overnight, followed by reduction in 1M aqueous dimethylamine borane or sodium borohydride for 6 hrs. Copper metallization was confirmed by Energy Dispersive X-ray Analysis, which was done using a FEI XL30 FEG-SEM equipped with a PGT-IMIX PTS EDX system.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

All references cited herein are incorporated fully by reference.

What is claimed is:

1. A composition comprising a polymer substrate having a surface comprising a donor-electron pair on the surface capable of coordinating with a metal alkoxide, and a continuous oxide adhesion layer formed by a matrix of individual molecules that are chemically bonded and linked to each other, wherein the continuous oxide adhesion layer comprises a metal oxide bonded directly adjacent to the surface by vapor deposition or immersion deposition, and wherein the continuous oxide adhesion layer comprises a metal alkoxide, the metal alkoxide having been subjected to treatment by one or more of the group consisting of pyrolysis, microwaving, complete hydrolysis and partial hydrolysis.

2. The composition of claim 1, wherein the metal alkoxide is zirconium tetra(tert-butoxide).

3. The composition of claim 1, wherein the metal is a Group 3-6 or Group 13-14 metal.

4. The composition of claim 1, wherein the alkoxide is selected from the group consisting of ethoxide, propoxide, iso-propoxide, butoxide, iso-butoxide, tert-butoxide and fluorinated alkoxide.

5. The composition of claim 1, comprising an additional material selected from an organic, metallic, organometallic or inorganic compound disposed on the oxide adhesion layer.

6. The composition according to claim 5 wherein the additional material comprises an organic compound sufficiently acidic to react with a metal oxide or alkoxide.

7. The composition according to claim 5 wherein the additional material comprises an organic compound selected from the group consisting of a carboxylic, phosphonic, phosphoric, phosphinic, sulfinic, sulfonic and hydroxamic compound.

8. The composition according to claim 5 wherein the additional material comprises a metal or metallic compound selected from the group consisting of Cu, Ag, Au, Al, Ni, Rh, Pd or Pt or a salt thereof.

9. The composition according to claim 5 wherein the additional material comprises an organometallic compound that can react with an oxide, alkoxide, hydroxide, hydroxyl or π-bond.

10. The composition according to claim 5 wherein the additional material comprises an inorganic compound.

11. The composition according to claim 5 wherein the additional material is selected from the group consisting of silanes, siloxanes, carboxylates, phosphonates, alkenes, alkynes, alkyl halides, epoxides, carboxylic esters, amides, phosphonate esters and imides.

12. The composition according to claim 5 comprising a deposited additional material that has been subjected to heat or microwave treatment, wherein the additional material is a metal, an inorganic compound, a flexible circuit device or one or more polymers.

13. The composition according to claim 1 wherein the adhesion layer is functionalized to elicit cell attraction and the adhesion layer comprises a material selected from an organic acid, nucleic acid, protein, and peptide.

14. The composition according to claim 1 wherein the adhesion layer is functionalized for bioresistance.

15. The composition according to claim 14 wherein the adhesion layer comprises at least one PEGylated region.

16. The composition according to claim 1 wherein the adhesion layer is disposed on the substrate in a pattern or micropattern.

17. The composition of claim 1, wherein the polymer is selected from the group consisting of polyamides, polyurethanes, polyureas, polyesters, polyketones, polyimides, polysulfides, polysulfoxides, polysulfones, polythiophenes, polypyridines, polypyrroles, polyethers, silicones, polysiloxanes, fluoropolymers, amides, imides, polypeptides, polyethylene, polystyrene, polypropylene, glass reinforced epoxies, liquid crystal polymers, thermoplastics, bismaleimide-triazine (BT) resins, benzocyclobutene polymers, Ajinomoto Buildup Films (ABF), dielectric films, low coefficient of thermal expansion (CTE) films of glass and epoxies, and composites including these polymers.

18. The composition of claim 1, wherein the polymer is selected from the group consisting of polyethylene terephthalate (PET), polyetheretherketones (PEEK), and nylon.

19. A device selected from the group consisting of a cardiovascular implant, a vascular implant, a tissue scaffold device, an orthopedic implant, a spine implant, a dental implant, a maxillofacial implant and a general surgical device comprising the composition according to claim 1.

20. A composition comprising a polymer substrate comprising a surface comprising a donor-electron pair on the surface capable of coordinating with a metal alkoxide, the polymer substrate selected from the group consisting of PET, PEEK, PEKK, polyamides, polyurethanes, polyureas, polyesters, polyketones, polyimides, polysulfides, polysulfoxides, polysulfones, polythiophenes, polypyridines, polypyrroles, polyethers, silicones, polysiloxanes, polysaccharides, fluoropolymers, amides, imides, polypeptides, polyethylene, polystyrene, polypropylene, glass reinforced epoxies, liquid crystal polymers, thermoplastics, bismaleimide-triazine (BT) resins, benzocyclobutene polymers, Ajinomoto Buildup Films (ABF), dielectric films, low coefficient of thermal expansion (CTE) films of glass and epoxies, and composites including these polymers, and a continuous oxide adhesion layer formed by a matrix of individual molecules that are chemically bonded and linked to each other, wherein the continuous oxide adhesion layer comprises a metal oxide bonded directly adjacent to a surface of the polymer substrate by vapor deposition or immersion deposition, and wherein the continuous oxide adhesion layer comprises a metal alkoxide, the metal alkoxide having been subjected to treatment by one or more of the group consisting of pyrolysis, microwaving, complete hydrolysis and partial hydrolysis.
